(19) United States
(12) Patent Application Publication
(10) Pub. No.: US 2024/0423627 A1
(43) Pub. Date: Dec. 26, 2024

(54) DEVICES, SYSTEMS, AND METHODS FOR TREATING VOLUME OVERLOAD

(71) Applicant: The Board of Trustees of the Leland Stanford Junior University, Stanford, CA (US)

(72) Inventors: Stacie Arechavala, Portola Valley, CA (US); Kevin Bui, Portola Valley, CA (US); Ankit Garg, Portola Valley, CA (US); Paola Calvachi Prieto, Portola Valley, CA (US)

(21) Appl. No.: 18/829,086

(22) Filed: Sep. 9, 2024

Related U.S. Application Data
(63) Continuation of application No. PCT/US23/15213, filed on Mar. 14, 2023.
(60) Provisional application No. 63/269,342, filed on Mar. 14, 2022.
(60) Provisional application No. 63/362,997, filed on Apr. 14, 2022.

(51) Int. Cl. A61B 17/12; A61B 90/00

(52) U.S. Cl. CPC A61B 17/12109; A61B 17/12031; A61B 90/39; A61B 2017/1205; A61B 2017/12127; A61B 2090/3933; A61B 2090/3966

(57) ABSTRACT

Devices, systems, and methods for treating volume overload are disclosed herein. According to some examples, the present technology includes a method comprising delivering a distal portion of an elongate shaft to a treatment site within a vein of an upper chest of a patient, where the treatment site is between a superior vena cava and a left axillary vein. The method can include expanding an occlusive member at the treatment site proximal of an opening to a thoracic duct, thereby blocking blood flow proximal of the occlusive member. The method can further include injecting a contrast agent at the treatment site distal of the occlusive member such that the contrast agent flows retrograde into the thoracic duct.

### CROSS-REFERENCE TO RELATED APPLICATION(S)

The present application is a continuation of International Application No. PCT/US2023/015213, filed Mar. 14, 2023, which claims the benefit of priority to U.S. Provisional Application No. 63/269,342, titled DEVICES, SYSTEMS, AND METHODS FOR IMPROVING LYMPHATIC SYSTEM DRAINAGE, filed Mar. 14, 2022, and to U.S. Provisional Application No. 63/362,997, titled SYSTEMS AND METHODS TO IMPROVE LYMPHATIC FLOW, filed Apr. 14, 2022, the disclosures of which are incorporated by reference herein in their entireties.

### TECHNICAL FIELD

The present technology relates to devices, systems, and methods for treating volume overload. In particular, the present technology is directed to treating volume overload by improving lymphatic system drainage.

### BACKGROUND

Chronic and acute congestive heart failure (CHF) generally occurs when the heart is incapable of circulating an adequate blood supply to the body. This is typically due to inadequate cardiac output, which has many causes. In CHF decompensation fluids back up in a retrograde direction through the lungs and venous/lymphatic systems throughout the body, causing discomfort and organ dysfunction. Many diseases can impair the pumping efficiency of the heart to cause CHF, such as coronary artery disease, high blood pressure, and heart valve disorders.

In addition to fatigue, one of the prominent features of CHF is the retention of fluids within the body. Commonly, gravity causes the retained fluid to accumulate to the lower body, including the abdominal cavity, liver, and other organs, resulting in numerous related complications. Fluid restriction and a decrease in salt intake can be helpful to manage the fluid retention, but diuretic medications are the principal therapeutic option, including furosemide, bumetanide, and hydrochlorothiazide. Additionally, vasodilators and inotropes may also be used for treatment.

While diuretics can be helpful, they are also frequently toxic to the kidneys and if not used carefully can result in acute and/or chronic renal failure. This mandates careful medical management while in a hospital, consuming large amounts of time and resources. Hence, the ability to treat fluid retention from CHF without the need for toxic doses of diuretics would likely result in better patient outcomes at substantially less cost.

Fluid retention is not limited only to CHF. Conditions such as organ failure, cirrhosis, hepatitis, cancer, and infections can cause fluid buildup near the lungs, referred to as pleural effusion. The space is lined by two thin membranes (the visceral and parietal pleura) that line the surface of the lungs and the inside of the chest wall. Normally, only a few teaspoons of fluid are located in this space so as to help the lungs to move smoothly in a patient's chest cavity, but underlying diseases can increase this amount. Patients with pleural effusion may need frequent draining directly via a guided needle and catheter introduced directly to the pleura. These procedures are expensive, traumatic, and require hospitalization.

Accordingly, there is a need for improved treatments for fluid buildup in the body.

### SUMMARY

The subject technology is illustrated, for example, according to various aspects described below, including with reference to FIGS. 1A-13. Various examples of aspects of the subject technology are described as numbered clauses (1, 2, 3, etc.) for convenience. These are provided as examples and do not limit the subject technology.

1. A method for treating volume overload, the method comprising:
delivering a distal portion of an elongate shaft to a treatment site within a vein of an upper chest of a patient, wherein the treatment site is between a superior vena cava and a left axillary vein;
expanding an occlusive member at the treatment site proximal of an opening to a thoracic duct, thereby blocking blood flow proximal of the occlusive member; and
injecting a contrast agent at the treatment site distal of the occlusive member such that the contrast agent flows retrograde into the thoracic duct.

2. The method of Clause 1, further comprising visualizing the thoracic duct under an imaging modality after injecting the contrast agent at the treatment site.

3. The method of Clause 2, wherein the imaging modality is fluoroscopy.

4. The method of Clause 2, further comprising advancing an elongate member into the thoracic duct after visualizing the thoracic duct.

5. The method of any one of the previous Clauses, further comprising expanding an implant within at least a portion of the thoracic duct.

6. The method of any one of the previous Clauses, further comprising dilating the thoracic duct after visualizing the thoracic duct.

7. The method of any one of the previous Clauses, further comprising dilating the thoracic duct.

8. The method of any one of the previous Clauses, wherein the method further comprises monitoring the pressure within the thoracic duct to guide dilation of the thoracic duct.

9. The method of any one of the previous Clauses, wherein the treatment site is a first treatment site, and wherein the method further comprises:
collapsing the occlusive member,
advancing the elongate shaft distally to a second treatment site, wherein the first treatment site is along a left brachiocephalic vein and the second treatment site is along a left subclavian vein,
expanding the occlusive member at second treatment site, and
injecting a contrast agent at the second treatment site distal of the occlusive member such that the contrast agent flows retrograde into the thoracic duct.

10. The method of any one of the previous Clauses, wherein the occlusive member is a first occlusive member expanded in the left brachiocephalic vein, and wherein the method further comprises expanding a second occlusive member in a left subclavian vein.

11. The method of Clause 10, wherein the second occlusive member is carried by the distal portion of the elongate shaft.

12. The method of Clause 10, wherein the elongate shaft is a first elongate shaft, and wherein the second occlusive member is carried by the second elongate shaft.

13. The method of Clause 10, further comprising expanding a third occlusive member in the internal jugular vein.

14. The method of any one of the previous Clauses, wherein the treatment site is a first treatment site, and wherein the method further comprises:
collapsing the occlusive member,
advancing the elongate shaft distally to a second treatment site, wherein the first treatment site is along a left brachiocephalic vein and the second treatment site is along a left internal jugular vein,
expanding the occlusive member at a second treatment site, and
injecting a contrast agent at the second treatment site distal of the occlusive member such that the contrast agent flows retrograde into the thoracic duct.

15. The method of any one of the previous Clauses, wherein the occlusive member is a first occlusive member expanded in the left brachiocephalic vein, and wherein the method further comprises expanding a second occlusive member in a left internal jugular vein.

16. The method of Clause 15, wherein the second occlusive member is carried by the distal portion of the elongate shaft.

17. The method of Clause 15, wherein the elongate shaft is a first elongate shaft, and wherein the second occlusive member is carried by the second elongate shaft.

18. The method of Clause 1, wherein the occlusive member is shaped such that injection of the contrast agent is localized to a lymphovenous junction.

19. The method of any one of the previous Clauses, wherein the occlusive member comprises multiple lobes.

20. The method of any one of the previous Clauses, wherein the occlusive member is shaped such that, when expanded at the treatment site, the occlusive member has a first portion positioned in a left brachiocephalic vein and a second portion positioned in a left internal jugular vein.

21. The method of Clause 1, wherein the occlusive member is shaped such that, when expanded at the treatment site, the occlusive member has a first portion positioned in a left brachiocephalic vein and a second portion positioned in a left subclavian vein.

22. The method of any one of the previous Clauses, wherein the occlusive member is shaped such that, when expanded at the treatment site, the occlusive member has a first portion positioned in a left brachiocephalic vein, a second portion positioned in a left internal jugular vein, and a third portion positioned in a left subclavian vein.

23. A method for treating volume overload, the method comprising:
delivering a distal end portion of an elongate shaft to a treatment site within a vein of an upper chest of a patient, where the treatment site is between a superior vena cava and a left axillary vein;
creating a localized pressure gradient at the treatment site proximal of an opening to a thoracic duct, thereby blocking blood flow proximal of the occlusive member; and
injecting a contrast agent at the treatment site distal of the occlusive member such that the contrast agent flows retrograde into the thoracic duct.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIGS. 1A-1D illustrate common anatomical variations in the location of the lymphovenous junction in accordance with the present technology.

FIG. 2A shows a treatment system configured in accordance with several examples of the present technology.

FIG. 2B is a cross-sectional view of a balloon catheter configured in accordance with several examples of the present technology.

FIG. 3 shows a treatment device configured in accordance with several examples of the present technology.

FIG. 4 shows an implant configured in accordance with several examples of the present technology.

FIGS. 5A-5C depict a method for visualizing a lymphovenous junction in accordance with several examples of the present technology.

FIG. 6 illustrates a treatment system with an occlusive member expanded near a lymphovenous junction and configured in accordance with several examples of the present technology.

FIG. 7 illustrates a treatment system with an occlusive member expanded near a lymphovenous junction and configured in accordance with several examples of the present technology.

FIG. 8 illustrates a treatment system comprising multiple occlusive members, shown positioned near a lymphovenous junction and configured in accordance with several examples of the present technology.

FIGS. 9A-9C depict a method for dilating the thoracic duct in accordance with several examples of the present technology.

FIG. 10 shows a steerable elongate shaft configured in accordance with several examples of the present technology.

FIG. 11 shows a distal portion of a treatment system comprising independently steerable elongate shafts configured in accordance with several examples of the present technology.

FIG. 12 shows an implant configured to extend from the thoracic duct into the venous circulation in accordance with several examples of the present technology.

FIG. 13 shows a clip configured in accordance with several examples of the present technology, shown positioned at the lymphovenous junction.

### DETAILED DESCRIPTION

A multitude of cardiovascular conditions can result in insufficient cardiac output, limiting the ability of the heart to circulate blood. This results in fluid building up in the venous system, increasing venous blood pressure. This increase of pressure inhibits drainage from the lymphatic system to the venous system and causes the lymphatic system to drain at a lower rate than it absorbs fluid, resulting in edema. Excess fluid typically accumulates within the abdomen and causes discomfort and organ damage in patients.

Historically, patients suffering from edema as a result of heart failure have managed the condition with dietary changes such as reduced sodium intake and diuretic medications. However, these methods alone are insufficient to treat edema and diuretics can become toxic to the kidneys when used in high doses. Recent efforts have focused on accessing the lymphatic system and manually removing fluid using a catheter. However, these methods have significant drawbacks. Acute internal drainage is a time-consuming process and must be done in-hospital, and only offers a temporary solution that does not address recurring congestion. Acute or chronic external drainage is also a complex process, requiring multiple pieces of specialized equipment. Furthermore, removing lymph from the body could result in the removal of important lymph components, causing detrimental health effects.

Accordingly, there is a need for a safe, long-term, and effective method for balancing fluid exchange between the venous and lymphatic systems in patients suffering from edema. Lymphatic vessels drain fluid called lymph from tissues throughout the body and return the fluid to the venous system through two collecting ducts, the thoracic duct (terminating almost always the left side of the body) and the right lymphatic duct (terminating on the right side of the body). An ideal location for accessing the lymphatic system is the lymphovenous junction (LVJ), the location where the thoracic duct drains into the venous system near the left subclavian and jugular veins. While the thoracic duct is one of the largest lymphatic vessels, with roughly 75% of all lymph in the body passing through it, the thoracic duct is still relatively small (about 2.5 mm in diameter in healthy individuals, and about 4-7 mm in diseased patients) and notoriously difficult to cannulate. In patients with CHF, venous pressure can make drainage of lymph at this location difficult. The rigid anatomy of the thoracic duct at the LVJ as well as the valve creates a functional bottleneck at this location, making it a target of interest for draining excess lymph.

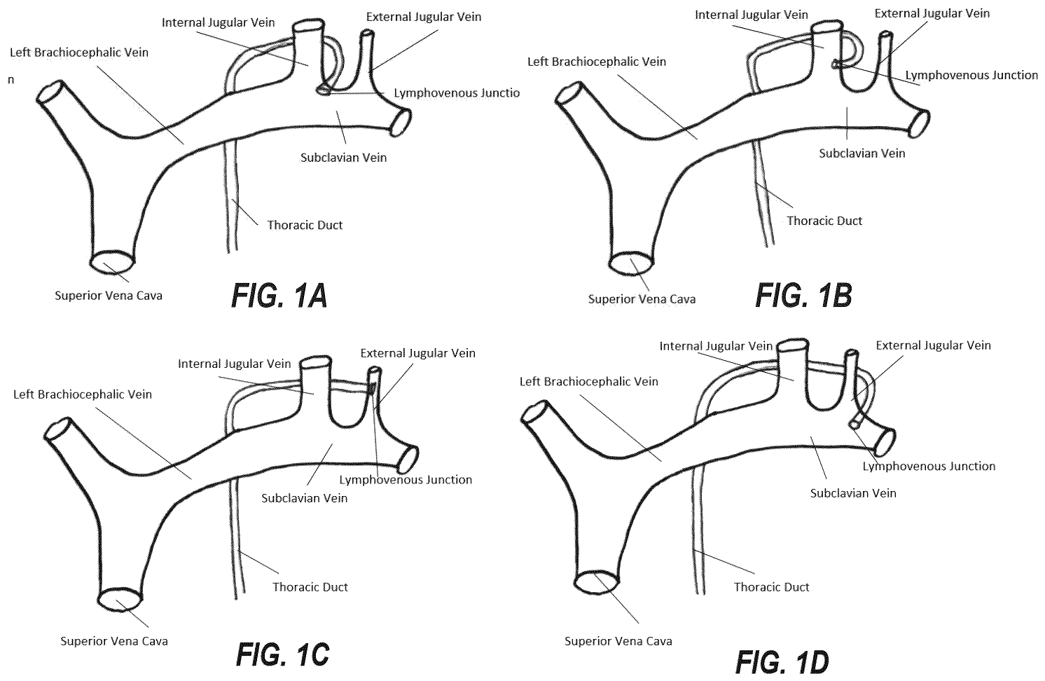

However, the precise location of the LVJ is not well understood in the field. This is in part because it can vary considerably between patients, as shown by FIGS. 1A-1D. In most patients, the TD joins the venous system at the venous angle (e.g., where the internal jugular vein and subclavian vein unite to form the brachiocephalic vein) (see FIG. 1A). It is also common for the LVJ to be located on the internal jugular vein (see FIG. 1B). In rare cases, the LVJ is located on the external jugular vein (see FIG. 1C), or along the subclavian vein, proximal to the external jugular vein (along the direction of blood flow) (see FIG. 1D). In a vast majority of cases, the TD terminates on the left side of the body, but may rarely terminate on the right side of the body, or bilaterally. The TD usually terminates as a single vessel, but it sometimes ends in bilateral vessels or as several terminal branches. In some cases, the TD may travel within the wall of the vein before communicating with the venous lumen.

In addition to the access challenges presented by its variable anatomy, the TD typically narrows at the LVJ (not depicted in the drawings) and has limited extensibility, making it difficult to cannulate. Some but not all TDs include a valve at the LVJ, presumably to regulate the flow of lymph into the venous circulation and prevent blood from entering the lymphatic system. The mechanism by which the valve does this is not well understood, and its role is made less clear by the fact that the valve is frequently absent, and when it is present it has a highly variable morphology.

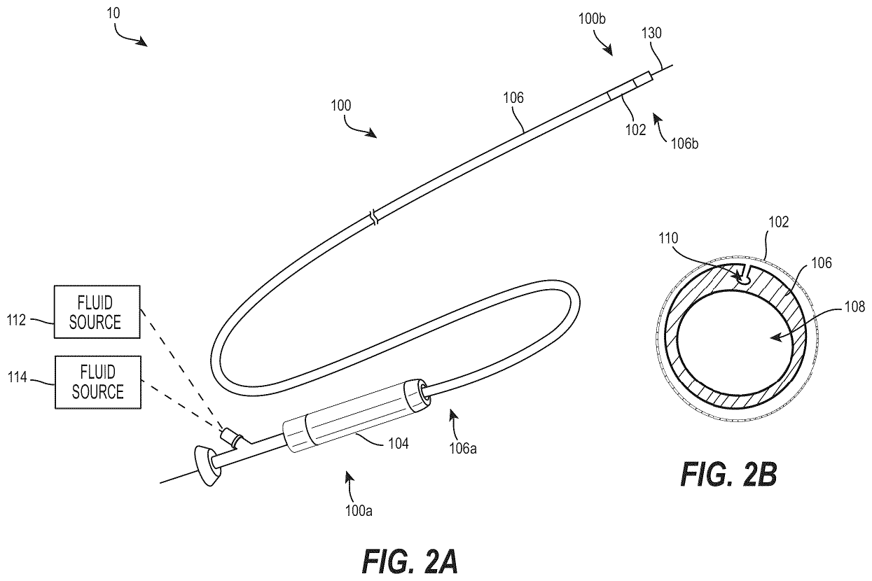

The devices, systems, and methods of the present technology are configured to identify and access the lymphovenous junction, in a location between the superior vena cava and a left axillary vein (e.g., the left brachiocephalic vein, the subclavian vein, the internal jugular vein, the external jugular vein, etc.), as well as increase flow from the TD into the draining vein. FIG. 2A, for example, illustrates a view of a treatment system 10 (or “system 10”) in accordance with several examples of the present technology. As shown in FIG. 2A, the system 10 can include a device 100 having a proximal portion 100a configured to be positioned external to the patient and a distal portion 100b configured to be intravascularly positioned within a vessel at a treatment site at or proximate an LVJ. As used herein, “vessel” refers to a blood vessel (such as a vein), the thoracic duct, and/or any other tissue connecting the terminal portion of the TD with the corresponding vein at the LVJ. The treatment device 100 can include a handle 104 at the proximal portion 100a, an elongate shaft 106 extending from a proximal portion 106a of the elongate shaft 106 at the handle 104 to a distal portion 106b of the elongate shaft 106, and an occlusive member 102 at the distal portion 106b of the elongate shaft 106. The occlusive member 102 may comprise a low-profile state for delivery to the treatment site (as shown in FIG. 2A) and an expanded state in which the occlusive member 102 is configured to engage at least a portion of the vessel wall to arrest the flow of fluids at the location of the occlusive member 102 and/or anchor the elongate shaft 106 with respect to a surrounding vessel.

In some examples, the occlusive member 102 comprises a balloon. For example, the occlusive member 102 can comprise a thin film adhered at its periphery to an outer surface of the elongate shaft 106. Introduction of a fluid (e.g., gas or liquid) to a space between the film and the surface of the elongate shaft 106 causes the film to stretch and expand radially away from the elongate shaft 106, thus inflating the balloon. Likewise, removal of fluid (e.g., via the application of negative pressure) causes the film to radially collapse down onto the elongate shaft 106, thus deflating the balloon. According to certain examples, the treatment device 100 includes two or more occlusive members 102 disposed on the elongate shaft 106 (see, for example, FIG. 6).

As shown in the axial cross-sectional view of the elongate shaft 106 in FIG. 2B, the elongate shaft 106 can comprise a generally tubular sidewall defining a lumen 108. The lumen 108 can be configured to receive one or more interventional devices, such as a guidewire, a visualization device, a catheter (such as a balloon catheter, an implant-loaded catheter, etc.), an implant and associated delivery system, and others. The lumen 108 may also be configured to be coupled to a fluid source to deliver fluid to the treatment site. In some examples, the elongate shaft 106 further includes an inflation lumen 110 having a proximal end at the proximal portion 100a of the device 100 and a distal end fluidly coupled to an interior region of the occlusive member 102, as shown in FIG. 2B. The treatment system 10 can include a first fluid source 112 (e.g., a syringe, a pump, etc.) configured to be fluidly coupled to a proximal end of the inflation lumen 110 to deliver fluid to/remove fluid from an interior region of the occlusive member 102 (e.g., to inflate/deflate the occlusive member 102).

The treatment system 10 can further include a second fluid source 114 (e.g., a syringe, a pump, etc.) configured to be fluidly coupled to the proximal portion 106a of the elongate shaft 106 to supply fluid (e.g., saline, contrast agents, therapeutic agents, etc.) to the treatment site (for example, to facilitate visualization of the TD). The fluid may be delivered either through the lumen 108 of the elongate shaft 106 or through a different lumen (not shown) within the elongate shaft 106. In the latter examples, the fluid delivery lumen can terminate distally at the distal face of the elongate shaft 106, or may terminate at one or more ports disposed along the sidewall of the elongated shaft 106. In some examples, the treatment system 10 includes a second, separate elongate shaft (not shown) for delivery of the fluid to the treatment site. The second elongate shaft can be configured to be coupled to the second fluid source 114, and can be configured to be slidably received through the lumen 108 of the elongate shaft 106 for positioning at the treatment site.

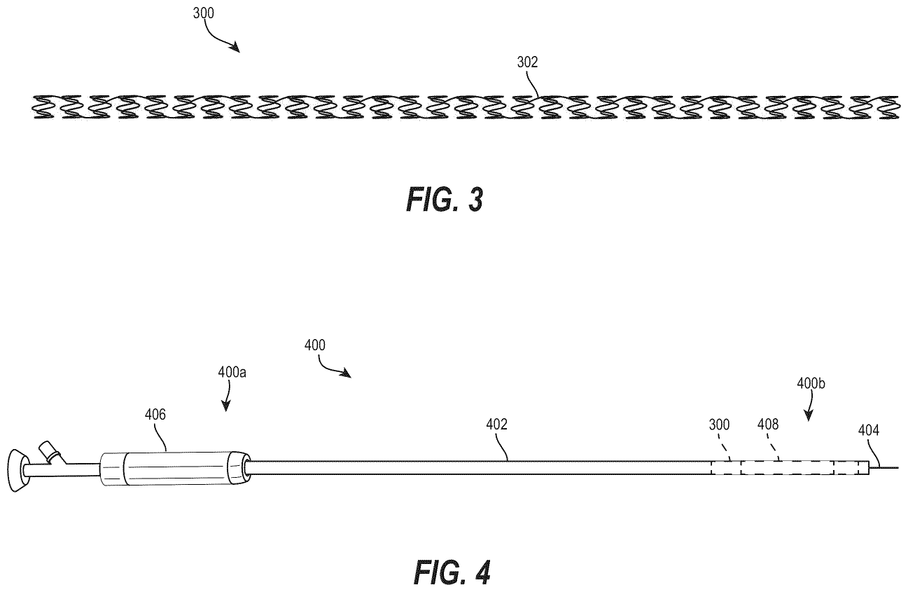

In some examples, the treatment system 10 further includes an expandable implant configured to be implanted partially or completely within the TD at the LVJ. For example, an implant 300 of the present technology and a delivery system 400 for positioning and deploying the implant 300 are shown in FIGS. 3 and 4, respectively. In some examples, the implant 300 comprises an open-form stent comprising a filament 302 (e.g., a wire, a strand, etc.) wrapped around a longitudinal axis of the stent such that no portion of the filament 302 crosses over itself. The flexibility and conformability provided by the open-form configuration can be beneficial for placement within the smaller and more delicate anatomy of the lymphatic vessels (as compared to blood vessels). According to some aspects of the technology, the implant comprises a weave and/or a braid formed of a plurality of braided filaments, or a single filament interwoven with itself, or a laser-cut stent comprising a plurality of interconnected struts and spaces (e.g., cells) between the struts. In any of the implant examples disclosed herein, the implant can be formed of a superelastic material or other resilient material that tends to resiliently assume an expanded configuration in the absence of a countervailing force. Additionally or alternatively, the implant may be configured for expansion by a balloon or other expandable structure.

Materials comprising the expandable implant could include but are not limited to shape-memory metals, soft platinum metal, stainless steel, titanium, titanium alloy, cobalt-chromium alloy, nitinol, platinum, other biocompatible metal alloys, alumina, bioglass, hydroxyapatite, medical-grade silicone, polyvinylchloride, polyethylene, polypropylene, polytetrafluoroethylene, polymethylmethacrylate, trimethylcarbonate, TMC NAD-lactide, or zirconia.

In some examples, the implant or any portion of the system may include a nonbiodegradable or biodegradable polymer coating to facilitate drug adhesion, drug release, biocompatibility, or modulate thrombogenicity within blood or lymphatic fluid. These polymers may include but are not limited to polylactic acid, polyglycolic acid, polylactic-co-glycolic acid, caproic acid, polyanhydride ester, salicylic acid, or sebacic acid. The system may also have a drug coating that may include but is not limited to sirolimus, tacrolimus, everolimus, leflunomide, M-prednisolone, dexamethasone, interferon r-1b, mycophenolic acid, mizoribine, cyclosporine, tranilast, paclitaxel (e.g., to prevent restenosis), actinomycin, methotrexate, angiopeptin, vincristine, mitomycin, statins, C-myc antisense, ABT-578, resten ASE, batimastat, prolyl hydroxylase inhibitors, halofuginone, C-proteinase inhibitors, probucol, BCP671, VEGF, estradiols, NO donor compounds, EPC antibodies, biorest, nintedanib, pirfenidone, and phenformin.

As shown in FIG. 4, the implant delivery system 400 comprises a proximal portion 400a configured to be positioned external to the patient and a distal portion 400b configured to be intravascularly positioned within a vessel at a treatment site at or proximate an LVJ. The delivery system 400 can include a handle 406 at the proximal portion 400a and an elongate shaft 402 (or “delivery shaft 402”) extending distally from the handle 406 to the distal portion 400b of the system 400. The delivery shaft 402 can define a lumen configured to slidably receive a guidewire 404 therethrough. As shown schematically in FIG. 4, the lumen may also be configured to contain the implant 300 in a low-profile or collapsed configuration. The delivery system 400 can optionally include an expansion member 408 slidably disposed within the delivery shaft 402, radially inwardly of the implant 300, and configured to be expanded at the treatment site to facilitate expansion of the implant 300 into apposition with an inner surface of the TD. Additionally or alternatively, the delivery system 400 can optionally include a dilation member (not shown for ease of viewing other features) disposed at an outer surface of the distal portion of the delivery shaft 402 and configured to be expanded at a treatment site within the TD to dilate the TD.

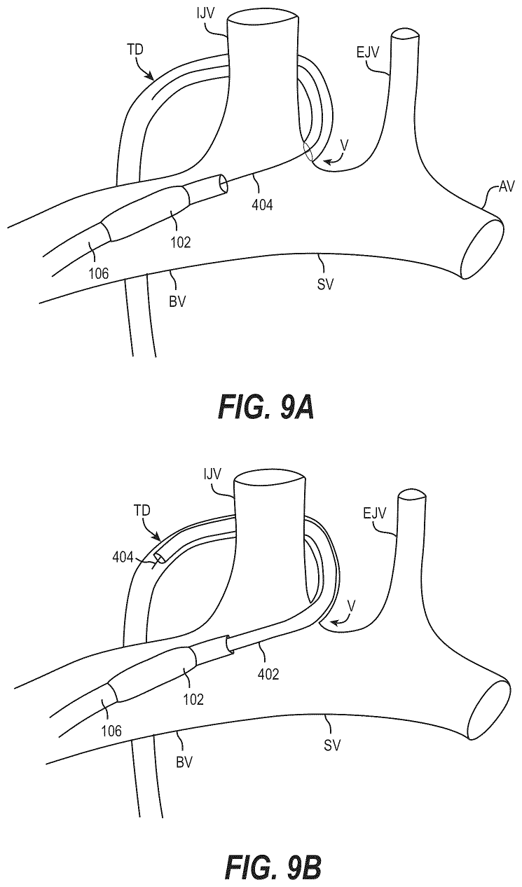

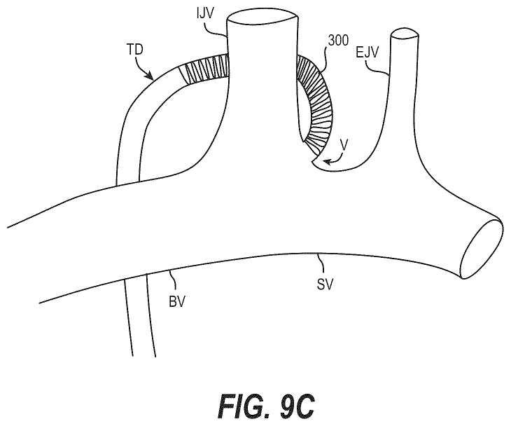

As discussed below with reference to FIGS. 9A-9C, the delivery shaft 402 can be configured to be slidably received within a lumen of the elongate shaft 105 (including lumen 108) to delivery to a treatment site within or near the TD. In some examples, a distal tip of the delivery shaft 402 can be softer than the rest of the length of the delivery shaft 402 to reduce vessel trauma. Additionally or alternatively, a distal tip of the delivery shaft 402 may be tapered to facilitate entry into the TD.

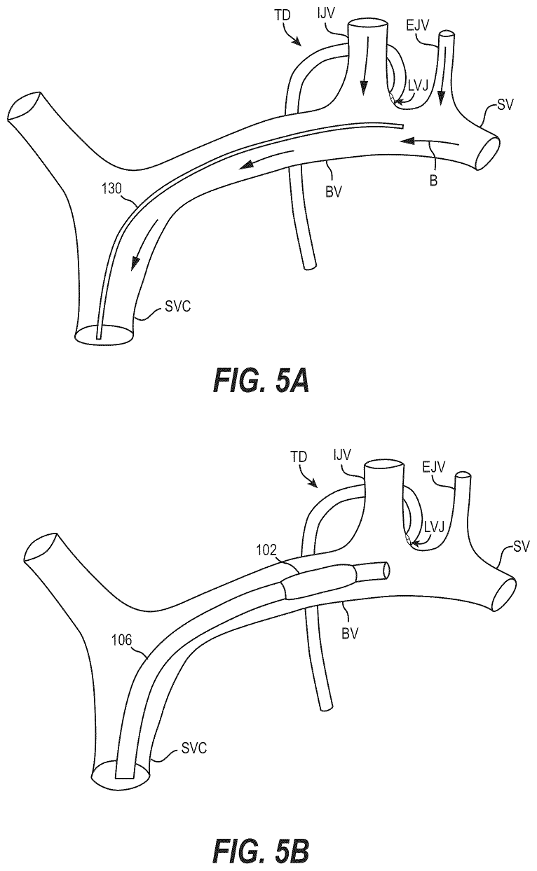

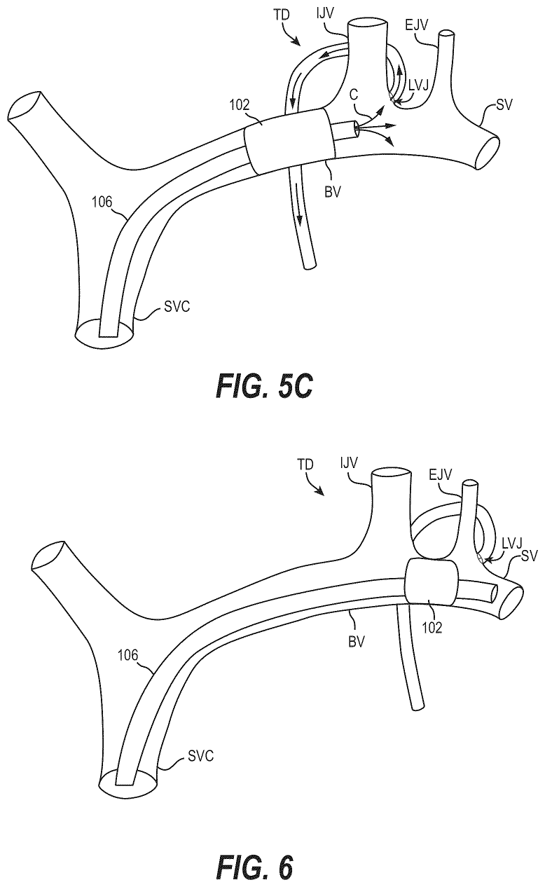

FIGS. 5A-5C illustrate a method of visualizing the LVJ using the treatment system 10 described with respect to FIGS. 2A and 2B. While FIGS. 5A-5C show the most common location of the LVJ (described with respect to FIG. 1A), the methods disclosed herein are not limited to a particular variant. Access to the venous system can be obtained percutaneously by inserting a guidewire 130 into a peripheral vein (e.g., femoral, basilic, cephalic, axillary, subclavian, internal jugular, or iliac veins) and advancing the guidewire 130 retrograde (e.g., against the flow of blood B) until a distal portion of the guidewire 130 is proximate the LVJ, such as within the brachiocephalic vein or subclavian vein (as shown in FIG. 5A). As previously mentioned, in the vast majority of cases the LVJ is on the patient's left side, so most likely the procedure will begin with placement of the guidewire 130 in the left brachiocephalic vein or left subclavian vein.

As depicted in FIG. 5B, the elongate shaft 106 may then be advanced over the guidewire 130 with the occlusive member 102 in its low-profile configuration. Positioning of the guidewire and/or elongate shaft 106 may be aided by imaging guidance, such as fluoroscopy, computed tomography, magnetic resonance imaging, ultrasound, intravascular ultrasound, or optical coherence tomography.

With the elongate shaft 106 in place, the occlusive member 102 may then be expanded into opposition with the venous wall at the treatment site (as shown in FIG. 5C), downstream of the LVJ. As the LVJ is most commonly at the juncture of the subclavian and internal jugular veins or along the internal jugular vein, in many cases the occlusive member 102 can be expanded into contact with the brachiocephalic vein, at least initially. The occlusive member 102 can additionally or alternatively be expanded within the subclavian vein between the internal jugular vein and the external jugular vein (as shown in FIG. 6), or within the subclavian vein across or distal to the external jugular vein. In any case, expansion of the occlusive member 102 blocks the flow of blood downstream of the occlusive member 102. In some examples, the occlusive member 102, the elongate shaft 106, or both can have one or more radiopaque markers to help guide positioning and/or deployment of the occlusive member 102.

Referring still to FIG. 3C, a contrast agent C can be delivered to the treatment site while the occlusive member 102 remains expanded and occluding flow. The guidewire 130 remains in place or may be withdrawn while the contrast agent C is injected. The localized pressure gradient created by the expanded occlusive member 102 causes a temporary backflow of blood into surrounding vessels (including the TD) such that the contrast agent C enters the TD. In FIG. 3C, the contrast agent C is shown being delivered through a lumen (such as lumen 108, or another lumen) of the elongate shaft 106 that terminates at the distal end of the elongate shaft 106. In other examples, the contrast agent C may be delivered through one or more side ports of the elongate shaft 106. In such examples, the contrast agent C may be delivered simultaneously at different angles through multiple side ports to increase the probability of directing the contrast agent C into the TD. In some examples, the contrast agent C may be delivered through a separate elongate shaft 106 that is delivered through or separately of the elongated shaft 106. In some examples, a separate elongate shaft (not shown) is advanced to the treatment site from a different access location than the elongate shaft 106 with the occlusive member 102. For example, a separate elongate shaft for contrast delivery could be advanced to the treatment site via the axillary and/or subclavian veins, internal jugular vein, and/or external jugular vein. In some cases, the occlusive member 102 can be inflated/deflated multiple times to avoid prolonged occlusion of the vein.

During or immediately after injection of the contrast agent C, the treatment site can be visualized via an imaging modality, such as fluoroscopy, computed tomography, magnetic resonance imaging, ultrasound, intravascular ultrasound, or optical coherence tomography, etc., to identify the LVJ. If the LVJ and/or TD is not sufficiently visualized after injecting the contrast agent C, the occlusive member 102 can be collapsed (e.g., deflated), repositioned, and re-expanded as many times as needed to target a different LVJ location and ensure sufficient backflow into the TD. For instance, to target an LVJ at the external jugular vein (see FIG. 1C) or along the subclavian vein (see FIG. 1D), the occlusive member 102 may be expanded within the subclavian vein (e.g., upstream of the internal jugular vein). In some cases, the occlusive member 102 is expanded within the subclavian vein between the internal jugular vein and external jugular vein, as shown in FIG. 6.

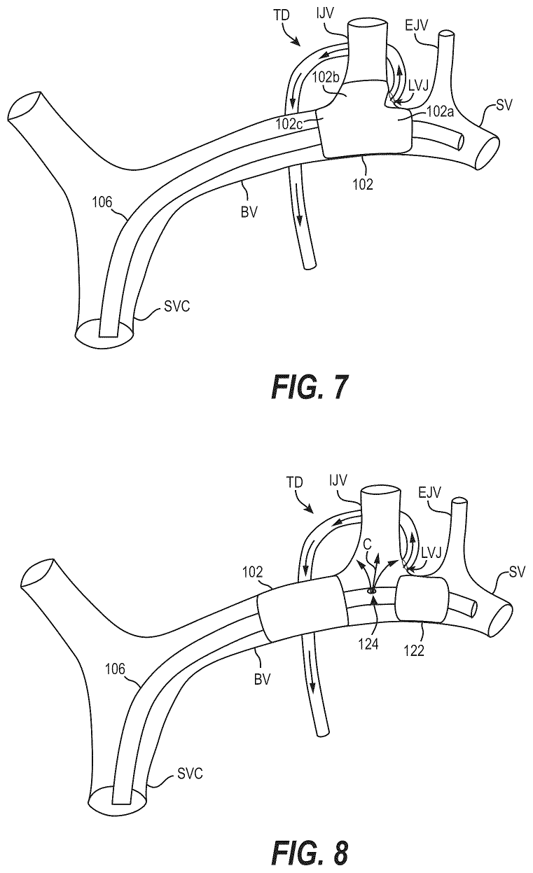

The occlusive member 102 can be sized and shaped to enhance the specificity of the generated pressure gradient at the treatment site. For example, in some examples the occlusive member 102 comprises multiple lobes. As shown in FIG. 7, the multiple lobes can be configured to complement the anatomy at the LVJ, for example at the venous angle. The occlusive member 102 can comprise a first lobe 102a configured to expand within the subclavian vein between the internal jugular vein and external jugular vein, a second lobe 102b configured to expand within the internal jugular vein, and a third lobe 102c configured to expand within the brachiocephalic vein. Other shapes and configurations are possible.

In some cases, it may be beneficial to utilize more than one occlusive member 102 to help localize the pressure gradient. For example, each of the occlusive members can be positioned so as to block nearby blood vessels, advantageously allowing more direct flow of contrast agent C into the TD. As shown in FIG. 8, in some examples the device 100 may include a first occlusive member 102 and a second occlusive member 122 disposed on the elongate shaft 106, longitudinally spaced apart from the first occlusive member 102. The first occlusive member 102 can be configured to be expanded into contact with the brachiocephalic vein while the second occlusive member 122 can be configured for expansion within the subclavian vein, upstream of the venous angle (as shown in FIG. 8). Positioning the multiple occlusive members proximal and distal of the potential LVJ site can enhance localization of the backflow, thereby improving visualization.

In some examples in which the device 100 includes multiple occlusive members, the elongate shaft 106 may include one or more ports disposed between the occlusive members. FIG. 8, for example, shows a side port 124 disposed along the elongate shaft 106 between the first and second occlusive members 102, 122.

In certain examples, multiple occlusive members 102 can be positioned at the treatment site on separate elongate shafts 106. For example, the elongate shaft 106 with the occlusive member 102 can be positioned in the brachiocephalic vein while another elongate shaft carrying another occlusive member can be positioned in the internal jugular vein. In some cases, the elongate shaft 106 with the occlusive member 102 can be positioned in the brachiocephalic vein while another elongate shaft carrying another occlusive member can be positioned in the subclavian vein. Additional variations are possible, including the use of three or more elongate shaft/occlusive members.

Following identification of the LVJ as described herein, the methods of the present technology proceed with accessing and dilating the TD (at the LVJ) to passively drain accumulated fluid into the venous system. FIGS. 9A-9C illustrate a method of dilating the thoracic duct using a treatment system 10 as described above. In some examples, the elongate shaft 106 may remain in place near the lymphovenous junction and serve as a sheath for through which additional equipment for lymphovenous access and intervention can be passed, such as guide wires for crossing the LVJ, a microcatheter for cannulating the LVJ, balloon catheters for dilating and stenting the LVJ, etc. As shown in FIG. 9A, the guidewire 404 of the implant delivery system 400 can be advanced through the elongate shaft 106 and distally across the LVJ (including across any valve(s) that may be present) and positioned within the TD such that a distal terminus of the guidewire 404 is positioned at or downstream of the descending portion of the TD. As shown in FIG. 9B, the delivery shaft 402 of the implant delivery system 400 can be advanced over the guidewire 130 (through the elongate shaft 106) and through the orifice at the LVJ and positioned within the TD. The delivery shaft 402 can then be withdrawn, leaving behind the implant 300, as shown in FIG. 9C. The implant 300 may self-expand upon removal of the constraints of the delivery shaft 402 and/or, as previously mentioned, the delivery system 400 can include an expansion member 408 that can be expanded and/or inflated (if a balloon) to facilitate expansion of the implant 300 into apposition with the wall of the TD. In some examples, prior to or while expanding the implant, an elongate member including an expansion element (such as a balloon or other device) can be positioned at the LVJ (for example, through the elongate shaft 106) and expanded to dilate the orifice at the LVJ. In some examples, a third elongate shaft is used to access the TD, and the delivery shaft 402 is advanced over the guidewire and through the third elongate shaft

In some examples, the occlusive member(s) 102 is collapsed (e.g., deflated) during placement of the implant and associated delivery systems. In some examples, the occlusive member may remain expanded (e.g., inflated) to help anchor the elongate shaft 106 and/or guide the implant delivery systems into the TD. The components of the implant delivery system may have a tendency to enter a larger branch of the vein such as the internal jugular vein, subclavian vein, external jugular vein. In some examples, the catheter or wire with balloon tip is inflated temporarily to block a side branch or multiple side branches, such that when an additional catheter or guidewire or microcatheter is inserted, it is blocked from entering the branches, and instead, is redirected to the LVJ of interest.

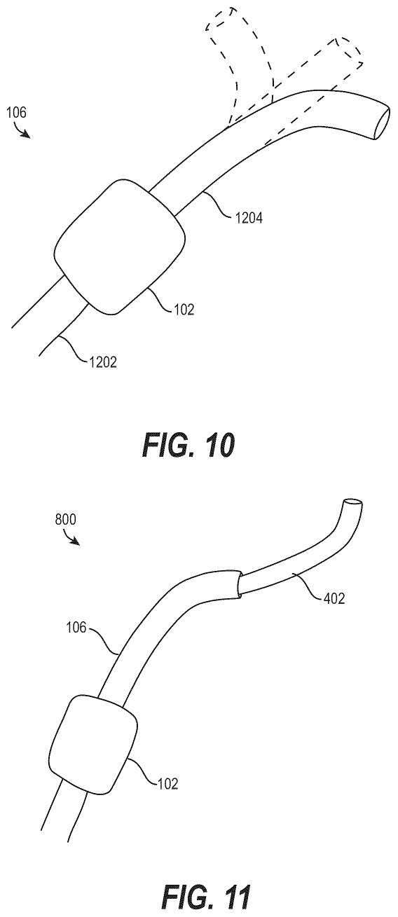

In some examples, the distal end of the elongate shaft 106 may have a steerable distal portion that facilitates delivery of equipment into the LVJ once visualized. For example, the elongate shaft 106 may include a plurality of pull-wires to control deflection of its sidewall, be configured for coupling to a robotic steering system, and/or having selectively deflectable portions activated by heating different portions of the sidewall. In some examples, the distal tip of the elongate shaft 106 (distal to the occlusion member 102) can selectively bent, flexed, and/or deflected to create a desired curvature that optimally orients the elongate shaft 106 (and any component passing therethrough) for cannulating the TD. FIG. 10, for example, shows the elongate shaft 106 having a first, non-deflectable portion 1202 and a second deflectable portion 1204. The first portion 1202 can extend from a location proximal of the occlusive member 102 to a location along the elongate shaft 106 that is aligned with or distal of a distal edge of the occlusive member 102. Accordingly, the entire second portion 1204 can be disposed distal of the occlusive member 102 such that deflection of the second portion 1204 does not cause the first portion 1202 of the elongate shaft 106 and occlusive member 102 to deflect. The second portion 1204 can have a bend radius, for example, of about 0.1 cm to about 3 cm.

The delivery shaft 402 can also have a steerable distal portion. According to some aspects of the technology, the elongate shaft 106 and the delivery shaft 402 may each be steerable, independent of one another, for example as shown in FIG. 11. This arrangement beneficially decouples steering and the ability to make a variety of advanced curves (e.g., such as S-shaped curves, reverse-shaped curves, etc.), thereby providing adequate catheter support for the cannulation of the TD and delivery of equipment across the junction. In some examples, the independent steerability can be controlled via a single handle with two actuators (e.g., knobs, dials, etc.).

In any of the examples disclosed herein, the elongate shaft 106 can have an outer diameter of at least 6 Fr, at least 8 Fr, at least 10 Fr, at least 12 Fr, at least 15 Fr, or at least 20 Fr.

In some examples, the catheter may be pre-shaped to cannulate the thoracic duct. This may be pre-shaped based on imaging before or during the procedure.

In some examples, the elongate shaft 106 (or other component of the treatment system) may include a pressure-sensing port in a region such that a measure of venous pressure can be obtained during inflation near the LVJ of interest. In some examples, once the venous pressure as a result of balloon inflation (e.g., expansion of the occlusive member 102) has increased to either 5 mmHg, 10 mmHg, 15 mmHg, 20 mmHg, 25 mmHg, 30 mmHg, 35 mmHg, 40 mmHg or higher, dye contrast is injected into the venous system. In some examples, the dye can be injected through a side port of the catheter (such as elongate shaft 106, or others), with an end-hole proximal to the balloon tip, such that the contrast flows into the venous region of interest. In some examples, a separate injection catheter or system can be used with the end-hole of the catheter placed near the region of interest. In some examples, after balloon inflation, the venous pressure increases near the LVJ, and as a result of venous pressure increasing above a level of that in the thoracic duct, contrast flows retrograde into the duct, opacifying the duct. In some examples, this may allow opacification of the terminal thoracic duct LVJ under fluoroscopy. In some examples, the catheter may contain a port that is placed proximal to the balloon, that allows passage of microcatheters or guidewires other systems into the LVJ with the use of a single access.

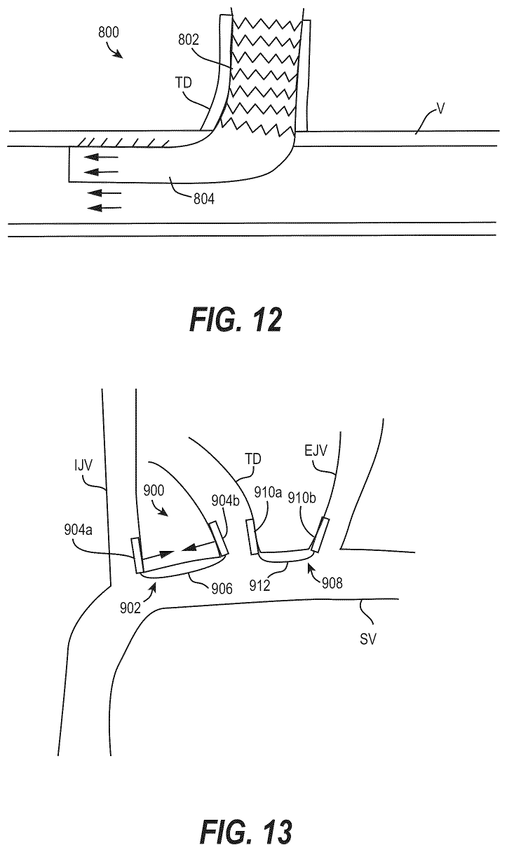

In some examples, a conduit (with or without a valve) is placed at the LVJ. As shown in FIG. 12, the a first portion 802 of a conduit 800 can be positioned within the TD and a second portion 804 can be positioned within the vein V. The second portion 804 can have one or more protrusions (e.g., anchors) configured to engage an inner surface of the vein wall to stabilize the second portion 804 in place against the wall. The second portion 804 can be more compliant than the first portion 802. All, some, or none of the conduit 800 may be covered. The conduit can comprise any of the implant 300 designs and materials disclosed herein. In some examples, the conduit 800 may comprise shape-memory metals, soft platinum metal, stainless steel, titanium, titanium alloy, cobalt-chromium alloy, other biocompatible metal alloys, alumina, bioglass, hydroxyapatite, medical-grade silicone, polyvinylchloride, polyethylene, polypropylene, polytetrafluoroethylene, polymethylmethacrylate, trimethylcarbonate, TMC NAD-lactide, or zirconia.

In some examples, a portion or all of the system may be compliant and compress as blood is passing (FIG. 12). This provides the advantage of passively pushing lymphatic fluid out of the conduit.

In some examples, the system 10 optionally includes a sensor for measuring a pressure across the LVJ. The sensor can be disposed, for example, at the distal portion of the elongate shaft 106 and/or delivery shaft 402. The sensor can be configured to measure the pressures across the lymphovenous junction simultaneously across a distance of up to 1 cm, 2 cm, 3 cm, 4 cm, 5 cm, 6 cm, 7 cm, 8 cm, 9 cm or 10 cm. The pressure sensing element may be placed such that it would simultaneously measure pressures distal and proximal to the valve, such as between the thoracic duct and the internal jugular or subclavian vein. The benefit of this would be to allow for precise location of pressure gradient measurements.

In some examples, the pressure sensing element may contain pressure sensing elements along the length of the catheter. In some examples, a system that allows for modulation of the local venous pressure at the thoracic duct outlet may be used if no gradient is initially observed while using the pressure sensing system. The benefit of this is to evaluate the true resistance to flow across the thoracic duct orifice and to identify a gradient that may be masked or missed due to elevations in downstream venous pressures which may occur in disease states such as heart failure or cirrhosis. In some examples, the system may consist of one or more pressure sensing elements and one or more occluding elements to modulate the venous outflow pressure. The pressure sensing element could be placed on a catheter, microcatheter, guidewire, balloon, stent, or valve. The occluding element can be placed on the pressure sensing element or be a separate component on a catheter, microcatheter, guidewire, balloon, stent, or valve. The occluding element may be but is not limited to a balloon or valves. The occluding element(s) may be deployed in the vein proximally, distally, or both to the thoracic duct outlet, such as in the subclavian vein, in the internal jugular vein, between the innominate vein and the thoracic duct outlet, or in between the thoracic duct outlet and the cephalic vein. The occluding element temporarily reduces the local venous outflow pressure. This has the benefit of removing the confounder of elevated venous pressures and enabling the true gradient to be measured across the lymphovenous junction. The pressure sensing element(s) may continuously measure pressure across the lymphovenous junction.

The orifice of the thoracic duct at the LVJ can be narrow relative to the diameter of the thoracic duct distal of the junction. Moreover, the tissue surrounding the orifice is generally inelastic. The treatment systems of the present technology can be configured to increase distensibility of the thoracic duct venous junction or junction orifice diameter to reduce resistance to flow and increase lymphatic flows across the thoracic duct venous junction. For example, in some examples, the treatment systems of the present technology may be configured to modify (e.g., disrupt, damage, inhibit, release and/or otherwise change the status quo of) smooth muscle in the wall of the junction and/or vein to reduce smooth muscle tension or constriction and thereby increase distensibility. The increased distensibility may advantageously lead to increased flow across the function, especially in cases of excess interstitial volumes or edema, such as heart failure or cirrhosis.

In some examples, a catheter can be passed transvenously from peripheral (leg/arm) or central (cervical neck vein, subclavian vein, cephalic vein) access and the LVJ in the cervical region is then cannulated. In some examples, upon cannulation, a guidewire is passed across the LVJ retrograde from the venous side into the duct. In some examples, a disruption element is passed over the guidewire and placed at the treatment site adjacent targeted smooth muscle at or near the orifice. In some examples, the sub-intimal layer of smooth muscle is targeted. In some examples, the treatment site is a specific region in which there is a band of smooth muscle that resembles a sphincter. In some examples, the element to damage smooth muscle can be placed at the LVJ or 1 mm, 2 mm, 3 mm, 4 mm, 5 mm, 6 mm, 7 mm, 8 mm, 9 mm, 10 mm distal or proximal to the junction.

In some examples, the disruption element is passed over the guidewire and placed at the treatment site. In some examples, the disruption element may comprise a balloon. In some examples, the balloon is placed at the treatment site and is inflated at pressures to damage the smooth muscle. These can include pressures of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or higher atm.

In some examples, the disruption element may comprise a balloon with sharpened protrusions configured to score the smooth muscle. In some examples, the balloon and sharpened protrusions may be sized to obtain a cutting depth of less than the depth between the intimal to adventitial layer that may aid in disrupting smooth muscle without causing perforation of the adventitial layer. In some examples, a balloon may comprise 1, 2, 3, 4, 5, 6, 7, 8, 9, 10, 11, 12 or more sharpened protrusions that are along the circumference of the balloon. In some examples, the cutting blades may be adjustable in size in order to adapt to the specific wall thickness of the layers of the LVJ. In some examples, the cutting balloon length may be adjusted after intravascular imaging to measure the layers of the wall of interest including the smooth muscle.

According to some methods, the treatment system is configured to perform a circumferential dissection of the intimal wall extending into the smooth muscle to modify the smooth muscle layer. In some examples, this may be performed with a rotational system with a diameter specifically set to disrupt the vessel wall diameter of interest. In some examples, the rotational system includes a knife or sharp edge that cuts the region of interest upon proximal/distal displacement of the system across the region of interest.

In some examples, the disruption element is a cutting element directly disposed on a catheter that may be passed over a guidewire to be delivered to the treatment site. The cutting element can be electrically or mechanically actuated to perform a cutting maneuver of the LVJ. In some examples, the cutting element is activated by radiofrequency, or ultrasonic energy. In some examples, the cutting element is placed on a catheter at one or more locations circumferentially along the outer surface of the catheter. In some examples, the catheter can be rotated to rotate the cutting element and perform a cutting maneuver along all or a portion of the surrounding vessel wall. In these and other examples, there may be cutting elements disposed on the catheter circumferentially including but not limited to the 12, 1, 2, 3, 4, 5, 6, 7, 8, 9, 10, 11, and/or 12 o'clock position.

In some examples, the depth of the cutting element extruding from the catheter or balloon is adjusted to adapt to the desired depth of cut required to modify smooth muscle at the treatment site. In some examples, the depth of the cutting element may be based on the intravascular imaging of the LVJ. In some examples, intravascular imaging of the junction including imaging of the lymphatic vessel segments can performed with ultrasound, optical coherence tomography or intravascular echocardiography catheter, or intracardiac echocardiography (ICE) catheter.

In some examples, the disruption element can be disposed on a wire or catheter and may have an adjustable angle, so as to restrict the depth of cut of smooth muscle. This may have the added benefit of reducing perforation while still ensuring an adequate disruption of smooth muscle. In some examples, the cutting element depth or length disposed on a catheter may be adjusted from knobs or dials placed on the handle of a catheter externally of the patient.

In some examples, imaging of the LVJ with an ICE catheter, optical coherence tomography (OCT) catheter, or intravascular ultrasound (IVUS) catheter is performed simultaneously during use of the cutting element used to disrupt smooth muscle at the junction. Such imaging advantageously enables direct visualization of the cutting element for safety to reduce the risk of perforation. In some examples, imaging of the LVJ and movement of the guidewire, catheter, or cutting element may be co-registered in order to provide feedback on location and/or depth of the guidewire/catheter or cutting element.

In some examples, the smooth muscle at the LVJ is modulated by either stimulation or inhibition to cause either constriction or dilatation of the smooth muscle at the junction.

In some examples, the LVJ can be dilated to a diameter larger than its original state. As shown in FIG. 13, in some examples, a clip system 900 is used to pull open the TD. The system 900 can include a plurality of clips 902, 904 placed between (a) one or more sides of the wall of the thoracic duct aspect of the junction and (b) one or more sides of the vein wall, with tension placed to open up the junction. In some examples, the system comprises a single clip. In some examples, the clip has grasping claws 904a, 904b and 910a, 910b with a link 906, 912, respectively, extending therebetween. The claws enable anchorage of the clip(s) on the venous and lymphatic aspects of the vessels. In some examples, the clip can extend from the thoracic duct to the IJV, or from the thoracic duct to the EJV, or from the thoracic duct to the SV, or the thoracic duct to the innominate vein, or some combination thereof.

In some examples, catheterization of the terminal thoracic duct or other lymphovenous junctions may also be performed via percutaneous access of the cisterna chyli or the long thoracic duct in the mediastinum. The benefit of this would be to facilitate an alternative access if the thoracic duct cannot be accessed via a peripheral vein (anterograde).

In some examples, the catheter or other ancillary devices used for catheterization may have special markers for identification of positioning. This may be to facilitate positioning and visualization under imaging guidance, such as hyperechoic and hypoechoic markers for detection under ultrasound or radiolucent and radiopaque markers for detection under fluoroscopy.

In some examples, resistance to flow at the junction of the thoracic duct into the venous system may be measured after cannulation of the thoracic duct. The pressure gradient across the thoracic duct-draining vein junction (may be internal jugular or subclavian vein or innominate vein) may be measured by passing a pressure measuring element located at the end of an ancillary device, such as a guidewire or catheter, through to the distal (terminal) thoracic duct, at a distance of up to 1 cm, 2 cm, 3 cm, 4 cm, or 5 cm or more from the venous portion. While collecting pressure measurements, the pressure measuring element can be pulled back up to 1 cm, 2 cm, 3 cm, 4 cm, 5 cm, 6 cm, 7 cm, 8 cm, 9 cm, or 10 cm or more toward to the draining vein and across the lymphovenous junction. The pull-back speed of the pressure measuring element may be done at a rate to detect a pressure gradient with expiration and inspiration. The patient may also be asked to hold their breath after expiration or inspiration.

In some examples, the system is configured to dilate the lymphovenous junction if a resistance to flow at the lymphovenous junction is identified or if a larger lymphovenous junction is desired.

In some examples, if access to the lymphovenous junction has not been obtained, access is obtained percutaneously by inserting a needle and guidewire into a peripheral vein, such as the femoral, basilic, cephalic, axillary, subclavian, internal jugular, or iliac veins. The guidewire is passed through the vein and then a sheath is advanced over the guidewire. A catheter is directed to the region of the lymphovenous junction. This procedure may involve fluoroscopy, computed tomography, magnetic resonance imaging, ultrasound, intravascular ultrasound, or optical coherence tomography to provide the benefit of imaging guidance to facilitate navigation of the wire and catheters. Once the catheter is advanced to the desired position in or near the thoracic duct outlet, the guidewire may be removed. A microcatheter can then be passed through the main catheter, with an intraluminal diameter smaller than the main catheter of no more than 1 mm, 2 mm, 3 mm, or up to the width of the main catheter. The microcatheter may also be advanced over a guidewire that is passed first through the orifice to facilitate microcatheter access. If the anterograde approach is challenging, a guidewire can be advanced retrograde through the cisterna chyli, with a receiving snare catheter on the venous side of the thoracic duct. Further imaging may be done at the lymphovenous junction to identify stenosis, the intramural course, and luminal diameter. Once the guidewire is across the junction, a balloon catheter can be placed at the junction in which there is resistance to flow and inflated to a pressure that causes the diameter of the junction to increase. The benefit of this is to reduce resistance to flow at the junction. The pressure gradient can be measured before and after inflating the balloon catheter to measure the effect of the treatment.

In some examples, the dilating element may be in the form of a scaffold or stent. It may be placed at the lymphovenous junction to maintain patency and durability of the expanded lymphovenous junction. The scaffold or stent may extend into the thoracic duct, the draining vein, or both. Its properties may include being able to withstand transmural pressures of up to 5 mmHg, 10 mmHg, 15 mmHg, 20 mmHg, 25 mmHg, 30 mmHg, 35 mmHg, 40 mmHg, or 50 mmHg; having a coating to prevent obstruction; having a coating designed to prevent clot formation; or having a drug eluting element to prevent restenosis or clot formation. The scaffold or stent may have a diameter of up to 2 mm, 4 mm, 6 mm, 8 mm, 10 mm, 12 mm, 14 mm, 16 mm, 18 mm, 20 mm, 22 mm, 24 mm, 26 mm, 28 mm, or 30 mm.

In some examples, the dilating element may be in the form of a balloon inflated across the lymphovenous junction for up to 30 seconds, 45 seconds, 60 seconds, 90 seconds, 120 seconds, 150 seconds, 180 seconds, 240 seconds, 300 seconds, or 360 seconds. The balloon may have a length of up to 5 mm, 10 mm, 15 mm, 20 mm, 25 mm, 30 mm, 35 mm, 40 mm, 45 mm, or 50 mm. In some examples, the dilating element(s) may be coated with and/or elute or release over time a substance to prevent restenosis or repeat narrowing of the lymphovenous junction. This coating may be a third-party pharmacological or non-pharmacological substance.

In some examples, the dilating element may be coated with different substances for the venous and lymphatic sides of the element in order to reduce restenosis or clotting based on the difference in tissue property of the different vessels.

In some examples, the dilating element(s) may be in the form of a bridge or separator to maintain vessel patency. In some examples, the dilating element may have a proximal diameter that is narrower than the distal diameter, or vice versa. It may also have one or more artificial valves. The valve may be a mechanical, tissue bioprosthetic, monocuspid, multi-cuspid, or ball and cage design. It may be designed to open based on the transmural pressure difference across the valve or based on a phase of the respiratory cycle, such as inspiration or expiration.

In some examples, the orifice lymphovenous valve is removed to increase the flow across the thoracic duct and draining vein. The benefit of this would be to allow for passive and continuous draining, overcoming the closing of the valve during respiration or elevated venous pressures. The elements of this system may include a valve removal element and a collection element. The lymphovenous valve is removed by passing a guidewire across the valve, then deploying a valve removal element. The valve rupturing element may consist of a balloon, cutting balloon, radiofrequency ablation, cryoablation, or laser energy. A collection element is used to collect the ruptured valve and remove it from the body.

In some examples, in a case in which the thoracic duct has an intramural junction with the vein, in which the terminal duct sits within the end of the vein, a dilating element may be placed inside the junction in order to prevent transmural junction occlusion at times of elevated venous pressures such as in disease states like heart failure.

In some examples, the local venous outflow pressure at the thoracic duct outlet can be modulated by inserting 1 or more occluding elements such as a balloon catheter from independent access sites such as the jugular veins or other peripheral veins and guided to the thoracic duct outlet. The occluding elements can then be activated to cause a reduction in the local venous outflow pressure, and pressure measurements across the lymphovenous junction can be simultaneously measured during the period of occlusion in order to identify a gradient.

### CONCLUSION

Although many of the examples are described above with respect to systems, devices, and methods for treating heart failure by reducing volume overload, the present technology is applicable to other applications and/or other approaches, including treatment of edema and/or volume overload resulting from conditions other than CHF, such as weakened venous valves, kidney disease, lung disease, liver disease, thyroid disease, side effects of certain medications, pregnancy, immune system complications, and traumatic injuries. Moreover, the treatment devices, systems, and methods of the present technology can be used for visualizing and/or treating portions of the lymphatic system other than the TD, such as the right lymphatic duct. Moreover, other examples in addition to those described herein are within the scope of the technology. Additionally, several other examples of the technology can have different configurations, components, or procedures than those described herein. A person of ordinary skill in the art, therefore, will accordingly understand that the technology can have other examples with additional elements, or the technology can have other examples without several of the features shown and described above with reference to FIGS. 1A-13.

The descriptions of examples of the technology are not intended to be exhaustive or to limit the technology to the precise form disclosed above. Where the context permits, singular or plural terms may also include the plural or singular term, respectively. Although specific examples of, and examples for, the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific examples have been described herein for purposes of illustration, but that various modifications may be made without deviating from the technology. Further, while advantages associated with certain examples of the technology have been described in the context of those examples, other examples may also exhibit such advantages, and not all examples need necessarily exhibit such advantages to fall within the scope of the technology. Accordingly, the disclosure and associated technology can encompass other examples not expressly shown or described herein.

We claim:

1. A method for treating volume overload, the method comprising:
delivering a distal portion of an elongate shaft to a treatment site within a vein of an upper chest of a patient, wherein the treatment site is between a superior vena cava and a left axillary vein;
expanding an occlusive member at the treatment site proximal of an opening to a thoracic duct, thereby blocking blood flow proximal of the occlusive member; and
injecting a contrast agent at the treatment site distal of the occlusive member such that the contrast agent flows retrograde into the thoracic duct.

2. The method of claim 1, further comprising visualizing the thoracic duct under an imaging modality after injecting the contrast agent at the treatment site.

3. The method of claim 2, wherein the imaging modality is fluoroscopy.

4. The method of claim 2, further comprising advancing an elongate member into the thoracic duct after visualizing the thoracic duct.

5. The method of claim 4, further comprising expanding an implant within at least a portion of the thoracic duct.

6. The method of claim 2, further comprising dilating the thoracic duct after visualizing the thoracic duct.

7. The method of claim 1, further comprising dilating the thoracic duct.

8. The method of claim 1, wherein the method further comprises monitoring the pressure within the thoracic duct to guide dilation of the thoracic duct.

9. The method of claim 1, wherein the treatment site is a first treatment site, and wherein the method further comprises:
collapsing the occlusive member,
advancing the elongate shaft distally to a second treatment site, wherein the first treatment site is along a left brachiocephalic vein and the second treatment site is along a left subclavian vein,
expanding the occlusive member at second treatment site, and
injecting a contrast agent at the second treatment site distal of the occlusive member such that the contrast agent flows retrograde into the thoracic duct.

10. The method of claim 1, wherein the occlusive member is a first occlusive member expanded in the left brachiocephalic vein, and wherein the method further comprises expanding a second occlusive member in a left subclavian vein.

11. The method of claim 10, wherein the second occlusive member is carried by the distal portion of the elongate shaft.

12. The method of claim 10, wherein the elongate shaft is a first elongate shaft, and wherein the second occlusive member is carried by the second elongate shaft.

13. The method of claim 10, further comprising expanding a third occlusive member in the internal jugular vein.

14. The method of claim 1, wherein the treatment site is a first treatment site, and wherein the method further comprises:
collapsing the occlusive member,
advancing the elongate shaft distally to a second treatment site, wherein the first treatment site is along a left brachiocephalic vein and the second treatment site is along a left internal jugular vein,
expanding the occlusive member at a second treatment site, and
injecting a contrast agent at the second treatment site distal of the occlusive member such that the contrast agent flows retrograde into the thoracic duct.

15. The method of claim 1, wherein the occlusive member is a first occlusive member expanded in the left brachiocephalic vein, and wherein the method further comprises expanding a second occlusive member in a left internal jugular vein.

16. The method of claim 1, wherein the occlusive member is shaped such that injection of the contrast agent is localized to a lymphovenous junction.

17. The method of claim 1, wherein the occlusive member comprises multiple lobes.

18. The method of claim 1, wherein the occlusive member is shaped such that, when expanded at the treatment site, the occlusive member has a first portion positioned in a left brachiocephalic vein and a second portion positioned in a left internal jugular vein.

19. The method of claim 1, wherein the occlusive member is shaped such that, when expanded at the treatment site, the occlusive member has a first portion positioned in a left brachiocephalic vein and a second portion positioned in a left subclavian vein.

20. The method of claim 1, wherein the occlusive member is shaped such that, when expanded at the treatment site, the occlusive member has a first portion positioned in a left brachiocephalic vein, a second portion positioned in a left internal jugular vein, and a third portion positioned in a left subclavian vein.

21. A method for treating volume overload, the method comprising:
delivering a distal end portion of an elongate shaft to a treatment site within a vein of an upper chest of a patient, where the treatment site is between a superior vena cava and a left axillary vein;
creating a localized pressure gradient at the treatment site proximal of an opening to a thoracic duct, thereby blocking blood flow proximal of the occlusive member; and
injecting a contrast agent at the treatment site distal of the occlusive member such that the contrast agent flows retrograde into the thoracic duct.
